(19) United States
(12) Patent Application Publication
(10) Pub. No.: US 2015/0008187 A1
(43) Pub. Date: Jan. 8, 2015

(54) BLOOD-PRETREATING APPARATUS AND METHOD

(71) Applicant: ELECTRONICS AND TELECOMMUNICATIONS RESEARCH INSTITUTE, Daejeon-city (KR)

(72) Inventors: Kwanghyo CHUNG, Daejeon (KR); Yohan CHOI, Daejeon (KR); Gunyong SUNG, Daejeon (KR); Jintae KIM, Daejeon (KR); Choongi CHOI, Daejeon (KR)

(73) Assignee: ELECTRONICS AND TELECOMMUNICATIONS RESEARCH INSTITUTE, Daejeon-city (KR)

(21) Appl. No.: 14/252,250

(22) Filed: Apr. 14, 2014

(30) Foreign Application Priority Data
Jul. 5, 2013 (KR) 10-2013-0078847

(51) Int. Cl. A61M 1/36

(52) U.S. Cl. CPC A61M 1/36
USPC 210/691; 210/257.1

(57) ABSTRACT

Provided are a blood-pretreating apparatus and method for removing corpuscles and abundant albumin from whole blood by use of microbeads. The blood-pretreating apparatus comprises a filter unit, packed with microbeads, for filtering out corpuscles and albumin through the microbeads from a blood sample introduced thereto; and a plasma storage unit for storing plasma free of corpuscles and albumin after the filtration in the filter unit

### CROSS REFERENCE TO RELATED APPLICATION(S)

This application claims the benefit of Korean Patent Application No. 10-2013-0078847, filed Jul. 5, 2013, which is hereby incorporated by reference herein in its entirety.

### BACKGROUND OF THE INVENTION

1. Technical Field

The present invention relates to an apparatus and method for the pre-treatment of blood. More particularly, the present invention relates to a blood-pretreating apparatus and method for separating plasma from whole blood.

2. Description of the Related Art

Blood contains many proteins some of which are used as important biomarkers indicative of diseases or health conditions. In recent years, much development has been made of biochips in the form of strips designed to easily and quickly analyze biomarkers using small amounts of blood.

Blood consists essentially of blood corpuscles and plasma. Corpuscles include erythrocytes, leucocytes, and platelets, accounting for more than about 40% of the volume of blood while plasma is comprised of water, proteins, lipids, carbohydrates, and minerals.

A trace amount of proteins useful as biomarkers is present only in plasma. Accordingly, the application of plasma only, free of blood corpuscles, to biochips is advantageous in detecting protein biomarkers and can be done so with high sensitivity and high reproducibility. In this regard, various suggestions have been provided for separating plasma from blood on chips. For example, corpuscles are filtered through paper, glass fibers or microstructures on chips. In addition, corpuscle bias, separation and preparation are achieved using centrifugal force, electromagnetic force, and gravity.

However, there is still been a demand for faster, simpler and more efficient techniques of removing corpuscles from a trace amount of non-diluted whole blood.

Accounting for 50% or more of the amount of total plasma proteins, albumin may be highly apt to act as a potent noise in detecting biomarkers that are present in a trace amount. Hence, elimination of albumin contributes to the high sensitivity and reproducibility of biochips.

Almost nowhere are means of removing albumin on biochips disclosed in previous literature.

A related prior art is found in Korean Patent Application Publication No. 2013-0057720 (“Blood pre-treatment apparatus and Pretreatment method using the same”) describing that albumin is removed from whole blood in a short time to obtain plasma of low viscosity.

Korean Patent Application Publication No. 2013-0057720 suggests forcible hemagglutination by which corpuscles can be quickly removed while plasma is obtained in a short time at improved efficiency.

That is, Korean Patent Application Publication No. 2013-0057720 aims to obtain only plasma by inducing a blood sample to undergo hemagglutination after the removal of albumin

### SUMMARY OF THE INVENTION

Accordingly, the present invention has been made keeping in mind the above problems occurring in the prior art, and an object of the present invention is to provide an apparatus and a method for the pre-treatment of blood in which corpuscles and abundant albumin are simultaneously removed from whole blood by use of microbeads.

In order to achieve the above object, a blood-pretreating apparatus according to one preferred embodiment of the present invention comprises: a filter unit, packed with microbeads, for filtering out corpuscles and albumin through the microbeads from a blood sample introduced thereto; and a plasma storage unit for storing plasma free of corpuscles and albumin after the filtration in the filter unit

Preferably, the filter unit may be configured to filter out corpuscles as the blood sample flows through microvoids formed between the packed microbeads.

Preferably, the microbeads may be coated with a functional group designed to adsorb albumin to the microbeads, so that albumin is removed in the filter unit by being captured by the functional group of the packed microbeads.

Preferably, the filter unit may have a cavity structure comprising a blood inlet and a plasma outlet wherein the blood inlet is positioned above the plasma outlet in view of gravitational direction and is greater in area than the plasma outlet.

Preferably, the cavity structure may be of any one of a frusto-cone, a quadrangular frusto-pyramid, a stepped cylinder, and a triangular frusto-pyramid.

Preferably, the side wall extending from the blood inlet to the plasma outlet may form an angle of 45 to 90 degrees with regard to the horizontal face.

Preferably, the microbeads may be wetted in PBS (phosphate buffer solution) and then dried.

Preferably, the microbeads may be pressurized by a load.

Preferably, the microbeads may range in diameter from 50 μm to 200 μm.

Preferably, the filter unit may be packed with the microbeads which are homogeneous or heterogeneous in diameter or which are a mixture of microbeads with homogeneous and heterogeneous diameters.

Preferably, the microbeads may be any one of Reactive Blue2, Cibacron Blue 3G-A, and Cibacron Blue F3FA.

Preferably, the filter unit may further comprise a cover for immobilizing the microbeads.

Preferably, the filter unit and the plasma storage unit may be detachably fitted to each other.

Preferably, the blood sample may flow as it is driven by capillarity and gravity.

Preferably, the plasma storage unit may further comprise a biosensor for detecting a biomarker in the plasma.

Preferably, the plasma storage unit may further comprise an air vent for helping the plasma to flow.

Preferably, a hydrophilic surface treatment may be applied to the blood pre-treating apparatus.

According to another preferred embodiment, the present invention provides blood-pretreating method, comprising: loading a blood sample to a filter unit packed with microbeads, said blood sample being deprived of corpuscles and albumin by the microbeads; and storing plasma in a plasma storage unit, said plasma being obtained by removing corpuscles and albumin from the blood sample.

Having the above-illustrated structure, the apparatus of the present invention can remove corpuscles and albumin, simultaneously, from whole blood only by dripping a blood sample, and thus can be applied to a biochip for detecting a biomarker at high sensitivity and reproducibility.

In addition, the apparatus and method according to the present invention can make a significant reduction in the blood amount required for analysis and in the time taken for plasma separation, and are applicable to various types of biochips, thus being qualified for general purpose uses.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a cross sectional view illustrating the structure of a blood pre-treating apparatus according to one embodiment of the present invention;

FIG. 2 is a perspective view of a blood pre-treating apparatus according to one embodiment of the present invention;

FIG. 3 illustrates structures of the filter unit given in FIGS. 1 and 2;

FIG. 4 illustrates cover structures for immobilizing the microbeads given in FIGS. 1 and 2; and

FIG. 5 illustrates packing modes of the microbeads given in FIGS. 1 and 2.

### DESCRIPTION OF THE PREFERRED EMBODIMENTS

Below, a detailed description will be given of the blood-pretreating apparatus and method according to embodiments of the present invention with reference to the drawings. Prior to the detailed description, it should be noted that words and terms used in the specification and the claims must not be construed as only conventional or dictionary meanings. Thus, since the embodiments given in the specification and the structures shown in the drawings are only preferable embodiments, but cannot cover all the technical spirit of the present invention, it should be understood that there may be various equivalents and modifications alternative to the given embodiments at the time of the application of the present invention.

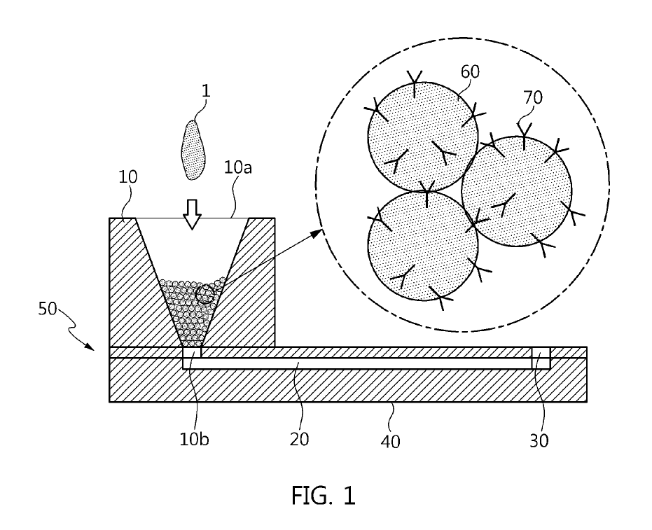

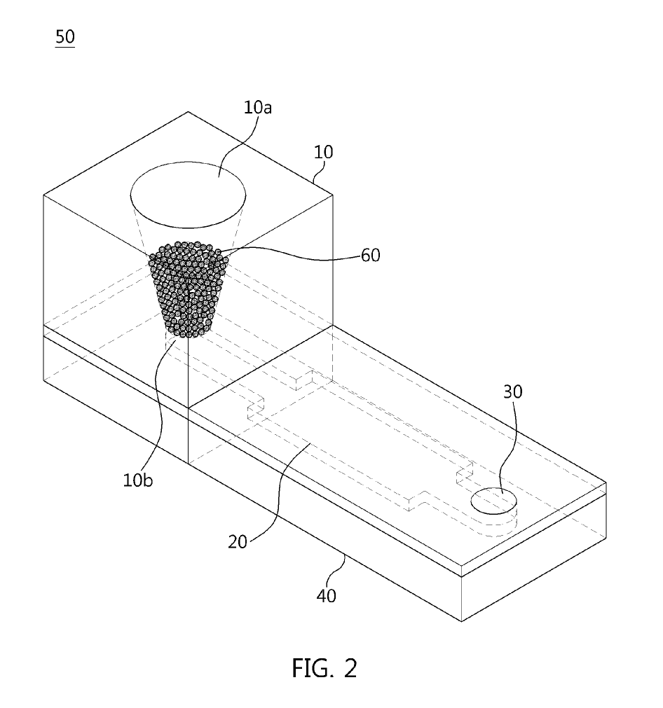

With reference to FIG. 1, a cross sectional view is provided for illustrating the structure of a blood pre-treating apparatus 50 according to one embodiment of the present invention while FIG. 2 is a perspective view of the blood pre-treating apparatus.

As shown, the blood pre-treating apparatus 50 according to an embodiment of the present invention comprises a filter unit 10, and a plasma storage unit 20.

The filter unit 10 is packed with predetermined microbeads 60 through which blood corpuscles and albumin are removed from a blood sample 1 (whole blood) introduced thereinto. Here, the filter unit 10 is formed on one side of a case body 40 while having a cavity structure comprising a blood inlet 10a to which whole blood 1 is introduced and a plasma outlet 10b from which plasma is released.

As mentioned above, the microbeads 60 are packed within the filter unit 10. They are coated with a functional group 70 designed to adsorb albumin to the surface of the microbeads 60. Hence, albumin is captured by the functional group 70 of the microbeads 60 so that it can be removed in the filter unit 10.

After the removal of corpuscles and albumin by the filter unit 10, the resulting plasma is stored in the plasma storage unit 20. Herein, the plasma storage unit 20 may be established within the case body 40. The one side of the plasma storage unit 20 is communicated with the plasma outlet 10b.

When the microbeads 60 are packed within the filter unit 10, non-uniform microvoids are formed among the microbeads. The microvoids are significantly smaller in size than the microbeads 60, and preferably designed to be as small as or smaller in size than corpuscles. To this end, the microbeads 60 preferably have a diameter of approximately 50 μm to 200 μm.

In order to pack the microbeads 60 at high density within the filter unit 10, a solution may be applied and then dried. For instance, a predetermined amount of dry microbeads 60 are added to the filter unit 10, wetted with PBS (phosphate buffer solution) and then dried to obtain a high-density packing structure. Alternatively, pressurization with a load may be adopted to realize a high-density packing structure. For example, a predetermined amount of dry microbeads 60 are added to the filter unit 10, and pressurized using a load which has a flat end.

In another embodiment, the filter unit 10 may be structured to be detachably fitted to the plasma storage unit 20. The detachable structures of the filter unit 10 and the plasma storage unit 20, although not shown, will sufficiently be understood to those skilled in the art on the basis of the structure and description of FIGS. 1 and 2. For the detachable structure, the filter unit 10 and the plasma storage unit 20 may be made of different materials, and then may be integrated with each other.

In the plasma storage unit 20, a biosensor (not shown) for detecting a biomarker in plasma may be established. Although it is not graphically illustrated, the establishment of the biosensor will be understood to those having ordinary skill in the art. In addition, the plasma storage unit 20 may be provided with an air vent 30 for facilitating plasma flow within the plasma storage unit 20. In FIGS. 1 and 2, the air vent 30 is positioned at a terminal region of the plasma storage unit 20 (that is, most distal to the plasma outlet 10b). However, the position may be changed as needed.

The microbeads 60 packed within the filter unit 10 may be commercial beads configured for absorbing albumin For the microbeads 60, for example, Reactive Blue2, Cibacron Blue 3G-A, Cibacron Blue F3FA, and a combination thereof may be used alone or in combination with various sub-synthetic compounds. However, the microbeads 60 are not limited to the above examples, and so long as it absorbs albumin, any microbead may be employed. The albumin within whole blood may be captured by the functional group 70 of the microbeads 60 within several seconds. A quantitative change of albumin can be monitored by, for example, SDS-PAGE (sodium dodecyl sulfate-polyacrylamide gel electrophoresis).

Preferably, the flow of the whole blood 1 proceeds only by dropping. That is, the driving force of the blood flow within the apparatus may be preferably obtained from capillarity and gravity, without an external physical force. In order for blood to smoothly move without an external force, a hydrophilic surface treatment for facilitating a capillary flow may be additionally applied to the blood pre-treating apparatus 50. For example, the apparatus may be surface oxidized by oxygen plasma technology, or may be coated with a surfactant or a protein. In addition or alternatively, the plasma storage unit 20 may be made of a hydrophilic material, such as glass, to facilitate capillary flow.

When dripped to the blood inlet 10a in the blood pre-treating apparatus 50 according to an embodiment of the present invention, the whole blood 1 flows through the microvoids formed among the packed microbeads 60, and comes out of the plasma outlet 10b to the plasma storage unit 20.

In this course, the whole blood is deprived of corpuscles by the microvoids, and albumin by the functional group of the microbeads, so that only plasma, free of corpuscles and albumin, is stored in the plasma storage unit 20.

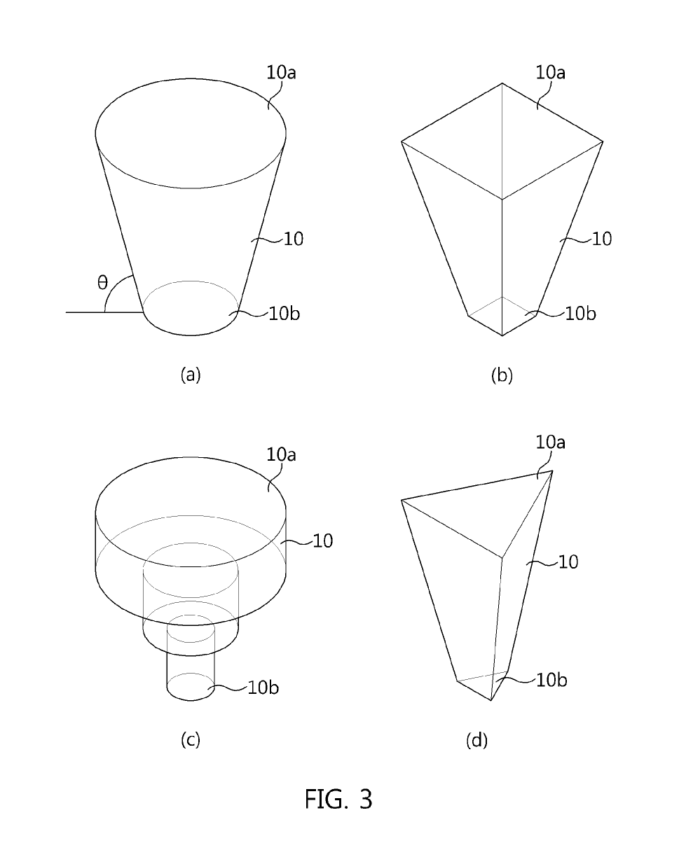

FIG. 3 illustrates various structures, particularly, cavity structures of the filter unit 10 given in FIGS. 1 and 2.

As shown in FIG. 3, the filter unit 10 may have various forms configured to allow the microbeads 60 to be packed therein, thereby serving to remove corpuscles and albumin simultaneously.

In order to obtain an improvement both in the migration of the whole blood in the apparatus and in the corpuscle removal efficiency, it is preferred that the blood inlet 10a be positioned above the plasma outlet 10b in view of gravitational direction and that the area of the blood inlet 10 is larger than that of the plasma outlet 10b. In this structure, the blood movement proceeds faster as the cross area is gradually reduced in the direction of blood flow, and the corpuscles can be removed more efficiently by the bottleneck phenomenon thus set.

As can be seen in FIGS. 3a, 3b, 3c and 3d, the cavity of the filter unit 10 may be modified to be of a frusto-cone, a quadrangular frusto-pyramid, a stepped cylinder, or a triangular frusto-pyramid. In these cavity structures, the side wall may preferably form an angle (θ) of 45 to 90 degrees with regard to the horizontal face.

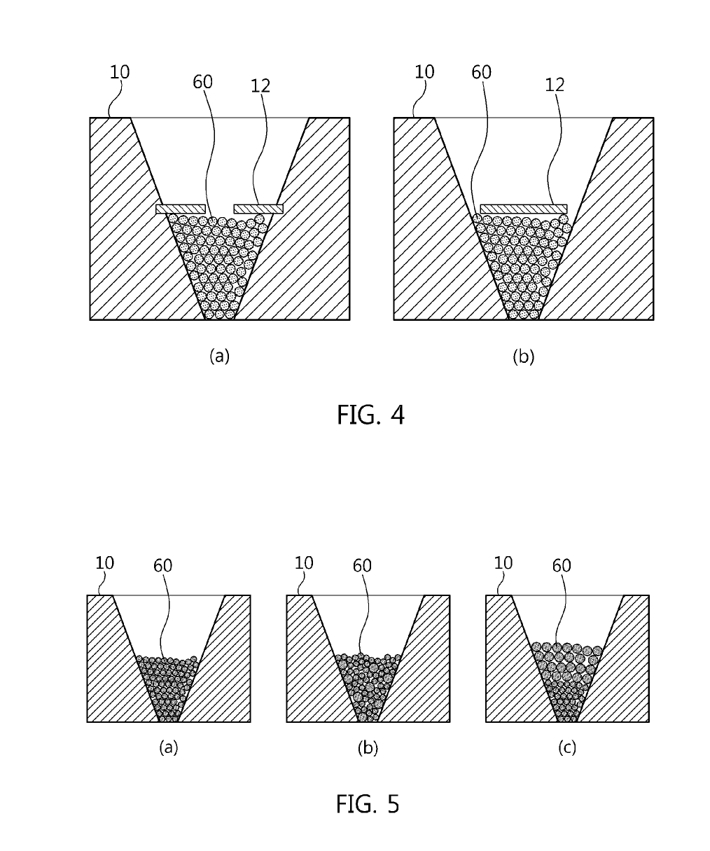

FIG. 4 illustrates cover structures for immobilizing the microbeads given in FIGS. 1 and 2.

With reference to FIG. 4, the filter unit 10 may be further provided with a cover 12 for immobilizing the microbeads 60. When the blood pre-treating apparatus 50 is upside down after the microbeads 60 are packed within the cavity of the filter unit 10, the microbeads do not maintain the packed condition, but escape towards the blood inlet 10a.

To prevent this, a cover 12 may be further established above the packed microbeads 60. The cover 12 may be fixed to the filter unit 10, as shown in FIG. 4a, or may be set to be immobilized on the microbeads 60.

FIG. 5 illustrates packing modes of the microbeads given in FIGS. 1 and 2.

Referring to FIG. 5, the microbeads 60 may be packed in various modes. The microbeads 60 may be homogeneous in diameter, as shown in FIG. 5a, or completely heterogeneous in diameter, as shown in FIG. 5b, or may be a mixture of microbeads with homogeneous and heterogeneous diameters. That is, only microbeads with a single diameter may be employed, or microbeads with different diameters are used in mixture.

Although the preferred embodiments of the present invention have been disclosed for illustrative purposes, those skilled in the art will appreciate that various modifications, additions and substitutions are possible without departing from the scope and spirit of the invention as disclosed in the accompanying claims.

[Description of Reference Numerals]

| | |
|---|---|
| 10: Filter Unit | 12: Cover |
| 20: Plasma Storage Unit | 30: Air vent |
| 40: Case Body | |
| 50: Blood Pretreating Apparatus | |
| 60: Microbeads | 70: Reactor |

What is claimed is:

1. A blood-pretreating apparatus, comprising:
a filter unit, packed with microbeads, for filtering out corpuscles and albumin through the microbeads from a blood sample introduced thereto; and
a plasma storage unit for storing plasma free of corpuscles and albumin after the filtration in the filter unit

2. The blood-pretreating apparatus of claim 1, wherein the filter unit is configured to filter out corpuscles as the blood sample flows through microvoids formed between the packed microbeads.

3. The blood-pretreating apparatus of claim 2, wherein the microbeads are coated with a functional group designed to adsorb albumin to the microbeads, so that albumin is removed in the filter unit by being captured by the functional group of the packed microbeads.

4. The blood-pretreating apparatus of claim 1, wherein the filter unit has a cavity structure comprising a blood inlet and a plasma outlet wherein the blood inlet is positioned above the plasma outlet in view of gravitational direction and is greater in area than the plasma outlet

5. The blood-pretreating apparatus of claim 4, wherein the cavity structure is of any one of a frusto-cone, a quadrangular frusto-pyramid, a stepped cylinder, and a triangular frusto-pyramid.

6. The blood-pretreating apparatus of claim 4, wherein a side wall extending from the blood inlet to the plasma outlet forms an angle of 45 to 90 degrees with regard to a horizontal face.

7. The blood-pretreating apparatus of claim 1, wherein the microbeads are wetted in PBS (phosphate buffer solution) and then dried.

8. The blood-pretreating apparatus of claim 1, wherein the microbeads are pressurized by a load.

9. The blood-pretreating apparatus of claim 1, wherein the microbeads range in diameter from 50 μm to 200 μm.

10. The blood-pretreating apparatus of claim 1, wherein the filter unit is packed with the microbeads which are homogeneous or heterogeneous in diameter or which are a mixture of microbeads with homogeneous and heterogeneous diameters.

11. The blood-pretreating apparatus of claim 1, wherein the microbeads are any one of Reactive Blue2, Cibacron Blue 3G-A, and Cibacron Blue F3FA.

12. The blood-pretreating apparatus of claim 1, wherein the filter unit further comprises a cover for immobilizing the microbeads.

13. The blood-pretreating apparatus of claim 1, wherein the filter unit and the plasma storage unit are detachably fitted to each other.

14. The blood-pretreating apparatus of claim 1, wherein the blood sample flows as it is driven by capillarity and gravity.

15. The blood-pretreating apparatus of claim 1, wherein the plasma storage unit further comprises a biosensor for detecting a biomarker in the plasma.

16. The blood-pretreating apparatus of claim 1, wherein the plasma storage unit further comprises an air vent for helping plasma to flow.

17. The blood-pretreating apparatus of claim 1, wherein a hydrophilic surface treatment is applied to the blood pre-treating apparatus.

18. A blood-pretreating method, comprising:
loading a blood sample to a filter unit packed with microbeads, said blood sample being deprived of corpuscles and albumin by the microbeads; and
storing plasma in a plasma storage unit, said plasma being obtained by removing corpuscles and albumin from the blood sample.

19. The blood-pretreating method of claim 18, wherein the microbeads are coated with a functional group which is designed to adsorb albumin to the microbeads so that the albumin is removed by being captured by the functional group of the packed microbeads, and wherein the filter unit is configured to filter out corpuscles as the blood sample flows through microvoids formed between the packed microbeads.

20. The blood-pretreating method of claim 18, wherein the microbeads range in diameter from 50 μm to 200 μm.
